(19) United States
(12) Patent Application Publication
(10) Pub. No.: US 2015/0132794 A1
(43) Pub. Date: May 14, 2015

(54) METHOD OF MANUFACTURING SAMPLE CONTAINERS

(71) Applicant: Sony DADC Austria AG, Salzburg (AT)

(72) Inventors: Georg Bauer, Salzburg (AT); Gottfried Reiter, Adnet (AT); Werner Balika, Seekirchen am Wallersee (AT); Christoph Mauracher, Salzburg (AT)

(73) Assignee: Sony DADC Austria AG, Salzburg (AT)

(21) Appl. No.: 14/407,839

(22) PCT Filed: Jun. 21, 2013
(86) PCT No.: PCT/EP2013/063062

§ 371 Date: Dec. 12, 2014

(30) Foreign Application Priority Data
Jun. 22, 2012 (GB) 1211147.2

(51) Int. Cl. G01N 33/50; G01N 33/53

(52) U.S. Cl. CPC G01N 33/5005; G01N 33/5308
USPC 435/34; 436/86; 422/547

(57) ABSTRACT

A method of manufacturing a batch of sample containers optimized for culturing particular animal cells or binding particular proteins under particular growth conditions. When a customer requires a batch of sample containers, an appropriate surface is first selected by a testing process. A test container is provided which has a surface subdivided into a plurality of test areas, for example in a two-dimensional square array. Each test area has a pre-defined combination of surface properties including a micro- or nano-structure and these properties are varied from test area to test area. The animal cells or protein of interest are then tested under the particular conditions to be used. The test area that provides the ‘best’ conditions is then selected. The manufacture of a production batch of containers is then carried out, wherein the test areas in the production batch all copy the ‘best’ surface structure selected in the test.

### CROSS REFERENCE TO RELATED APPLICATIONS

The present application claims the benefit of the earlier filing date of GB1211147.2 filed in the United Kingdom Intellectual Property Office on 22 Jun. 2012, the entire contents of which application is incorporated herein by reference.

### BACKGROUND

1. Field

The present disclosure relates to the manufacture of sample containers, for example containers for cell culturing or protein binding.

2. Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, is neither expressly or impliedly admitted as prior art against the present disclosure.

The traditional environment for in vitro growth of cells is a flat unstructured glass or plastics surface, for example the ubiquitous Petri dish. On the scale of a cell, such a surface is effectively infinite.

On the other hand, the in vivo environment for cell growth is a complex three-dimensional environment which imposes physical constraints on cell growth as well as providing a specific biochemical environment.

Over several decades a large body of research has been carried out in the general area of how surface properties, including micro- or nano-structure (that is to say, for example, structural features having one or more feature dimensions in the range of (say) 1-10 μm or 1-10 nm respectively), affect growth of cells on substrates, in particular initial adhesion, spreading, differentiation and alignment of animal cells. One review article is Théry, Journal of Cell Science 123, 4201-4213 (2010) [1].

Surface micro-structuring for cell culturing has included:
forming a grating of parallel grooves with different depths and pitches on polystyrene (PS) substrates [2],
forming a grating in polydimethylsiloxane (PDMS) with a feature height and width in the micrometre range where it was found that below a critical feature width of approximately 13 μm an otherwise cytophilic surface became cytophobic so that controlled microstructuring could be used to promote and inhibit cell growth on different areas of the substrate [3].
nanostructuring truncated pyramids on PS substrates [4],
moulding upstanding nanopillars on polycarbonate (PC) and polylactic acid (PLA) substrates [5], and
arranging TiO nanotubes on a surface [6]

The use of porous substrates has also been studied, such as foamed polymer [7].

Another known technique for promoting and affecting cell growth involves coating a substrate with physiological proteins, such as laminin, fibronectin or extracellular matrix (ECM) proteins on PS substrates. These techniques are disclosed in various publications from the Max Planck Institute for Polymer Research [8, 9, 10] and are also now commercially available from the company Intelligent Substrates of Baltimore, Md., USA [11]. Line and grid patterns are offered by this company. The substrate types offered are: glass coverslips, quartz coverslips, glass-bottom dishes and chambers, polycarbonate membranes, polydimethylsiloxane (PDMS), and PS. The proteins offered are: fibronectin, laminin, collagen I, collagen III, collagen IV, ECM proteins, fibrinogen, peptides and antibodies. Intelligent Substrates offers a “BioWrite Sampler” product which contains one of each of 15 different protein patterns in a grid representing the full set of commercially available patterns. Each pattern occupies a square millimeter with feature sizes on the tens of micrometre scale, with the overall patterned area being 4×4 mm.

Microstructuring of a substrate surface has also been used to increase phase contrast in phase contrast microscopy [12].

It is also known to treat or coat the substrate surface to affect cell growth, and in particular to promote initial adhesion or alignment to the substrate surface of cells held in solution. Some examples include:
use of a guanidine ligand and a primary amine to bond onto a surface and thereby promote cell culturing [13];
pre-treating a glass or plastic surface with ionene (an amine) to promote cell growth [14]; and
arranging a liquid crystal alignment layer on a polyimide substrate followed by a layer of liquid crystal for growing aligned neural cells [15].

A known form of pre-treatment for the surfaces of cell culture containers is plasma treatment which is used to adjust the degree of hydrophobicity or hydrophilicity of a substrate surface, which in turn is known to influence cell adherency. Corona discharge is a commonly applied form of plasma treatment.

From the above overview of the prior art, it will therefore be appreciated that there is a very large set of variables for defining surface properties in order to obtain desired cell culturing, wherein the exact combination of growth surface, particular cell line and particular growth environment interact in a complex way which is not necessarily predictable.

### SUMMARY

Respective aspects and features of the present disclosure are defined by the appended claims.

According to the present disclosure, a combinatorial approach is taken to selecting a suitable surface for culturing a particular cell line under particular culturing conditions for a specific set of studies, or for binding a particular protein under particular experimental conditions for a specific set of studies.

When a customer requires a batch of sample containers for culturing particular animal cells under particular growth conditions, or a batch of sample containers for binding a particular protein, an appropriate surface is first selected by a test process such as an automated test process involving (in part) a data processing apparatus.

According to this test process, a test culturing container is provided which has a substrate having a surface subdivided into a plurality of test areas, for example in a two-dimensional square array. Each test area has a pre-defined combination of surface properties including a micro- or nano-structure, wherein said micro- or nano-structure has at least one dimensional parameter whose value is different in different ones of the test areas so as to have different test areas that cover a range of values of the or each said dimensional parameter. Different test areas are thereby provided on the test container, which cover a range of values of one or more dimensional parameters. Particular cells, for example animal cells, of interest are then test cultured under the particular conditions to be used, or a particular protein of interest is bound under the particular conditions to be used, wherein the test is carried out simultaneously on each of the test areas. The cultured animal cells or bound proteins are then analyzed on a per test area basis. The analysis is used to select one of the test areas as providing suitable, preferably the most suitable, conditions for the particular animal cells or protein under the particular conditions tested.

The manufacture and or supply of a production batch of culturing containers is then carried out, wherein the test areas in the production batch have the surface properties selected from the test as being suitable, preferably the most suitable, for the intended use.

It is to be understood that both the foregoing general description and the following detailed description are exemplary, but are not restrictive, of the present technology.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows in schematic plan view a structured substrate of a culturing container;

FIG. 2 shows a schematic example 48 well microtiter plate of standard format together with its lid in which the base of each well has an array of differently structured surface portions;

FIG. 3 schematically shows the principal steps in a substrate manufacturing process;

FIG. 4 schematically shows some comparative results of contact angle measured on various structured surfaces formed on COP;

FIG. 5 schematically shows some comparative results of contact angle measured on various structured surfaces formed on PS; and

FIG. 6 is a schematic flowchart illustrating a manufacturing and/or supply process.

### DESCRIPTION OF THE EMBODIMENTS

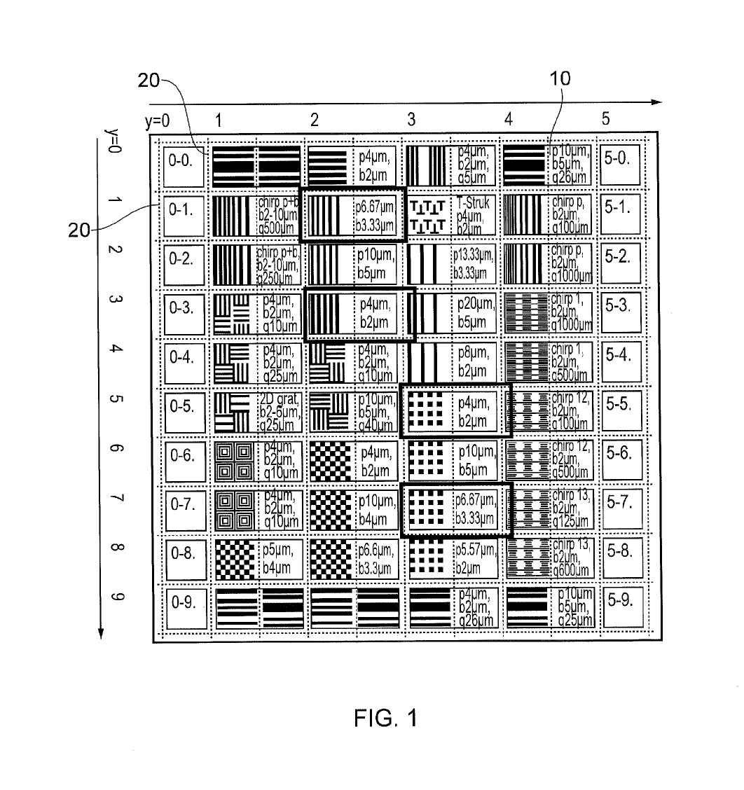

FIG. 1 shows in schematic plan view a structured substrate of a culturing container. The substrate has a surface subdivided into a plurality of test areas 10, 40 such areas being shown by way of example divided in a two-dimensional array of 4 columns and 10 rows according to the coordinate indices (x, y) where x ranges from 1 to 4 inclusive, and y ranges from 0 to 9 inclusive.

Each test area has a pre-defined combination of surface properties including a micro- or nano-structure. The structure in the test areas is labeled in FIG. 1 using the letters ‘p’ for periodicity or pitch and ‘b’ for breadth or width. The term “T-Struk” indicates a T-shaped formation, as viewed in plan view. Another parameter, such as a parameter q, may be defined, for example as a depth or height of the formations. Some of the structures have a chirp (a gradually varying periodicity or pitch across at least one dimension of the structure) and optionally also a varying width. The chirp may be defined by a parameter, which simply identifies that chirp pattern (as schematically illustrated) from other chirp patterns. Some of the structures are one dimensional in their variation comprising parallel ribs or grooves. Other ones of the structures are two dimensional in their variation, comprising arrays of protrusions or pits. The schematically illustrated structures are formed in the vertical dimension orthogonal to the substrate surface, for example being mesa-like, by approximately vertical sidewalls interconnecting a lower surface and an upper surface. With injection moulding, it may be appropriate for the sidewalls to be fabricated with a slight angle to vertical, for example between 80-90 degrees, in the non-overhanging sense. For each pattern there can be positive and negative versions of the same pattern which are related by the pits or depressions in one version being protrusions or pillars in the other complementary version. Other more complex patterns are also shown, such as: the swirl structures in column 1, rows 6 and 7; the “parquet flooring” structures in column 1, rows 3, 4 and 5 as well as column 2, rows 4 and 5; and the T-structures in column 3, row 1.

As just discussed, the structure features may be mesa-like, that is, formed by vertical or near vertical sidewalls with flat tops, but other structure features may be sawtooth or V-groove like, for example being formed by angled walls meeting at line ridges and troughs, for example at 45±15 degree angled walls. Alternatively, structure features may be formed from a combination of angular sidewalls with flat tops and/or bases.

The micro- or nano-structure has at least one dimensional parameter whose value is different in the different test areas with the general aim of having different test areas that cover a range of values of each varied dimensional parameter that is varied over the totality of test areas. Example dimensional parameters are feature periodicity in one or two directions in the plane of the surface (for example rib or trench separation, pit or pillar separation), feature width in one or two directions in the plane of the surface (for example rib or trench width, pit or pillar width) and feature height orthogonal to the plane of the surface (for example rib height, trench depth, pillar height or pit depth).

The size of the structure features is preferably scaled with the cell size of the cells to be cultured, in particular in the range between about the size of the cell (for example, between half and twice the cell size) and an order of magnitude less than the cell size (for example, between ¼, ⅕, 1/10 or 1/20 of the cell size), where cell size may be cell diameter for approximately circular section cells. For example, if the cell size is approximately 20 micrometres, the structure features may have dimensions in the range 2-10 micrometres, systematically varying one, two or three dimensions (such as one or more of width, height and vertical depth) from test area to test area over a range of several micrometres in steps of one micrometre.

Each test area is isolated from each other test area. This is done by having a relatively cytophobic area or strip 20 between each test area over which cell adhesion and growth is thus inhibited. This will stop, or at least inhibit, cells attaching and growing in between the designated test areas, and also colonies from spreading from one test area to an adjacent test area. The cytophobic strips can be formed of unstructured surface portions which are hydrophobic. Such unstructured surface portions can be substantially co-planar with the test areas. This can be termed a ‘virtual’ microtiter plate in that the separate test areas are analogous to wells in a microtiter plate. Alternatively, the test areas may be arranged in wells with each test area being isolated from each other test area by the test areas being formed as isolated wells recessed beneath an upper surface level with interconnecting sidewalls. The container can then have a microtiter plate format, or be a version thereof with shallow, perhaps extremely shallow, wells.

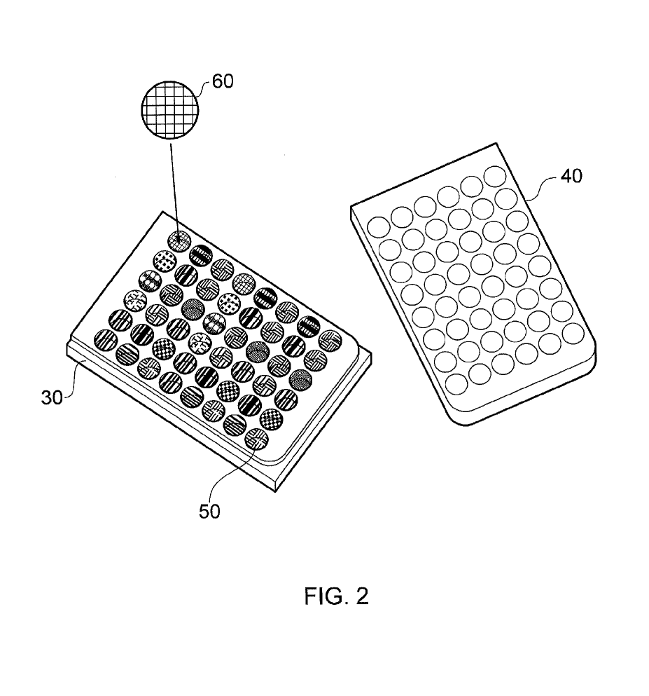

FIG. 2 shows an example 48 well microtiter plate (MTP) 30 of standard format together with its lid 40, in which the base of each well 50 has an array of differently structured surface portions. An example of such an array 60 is illustrated schematically in FIG. 2. In particular, in an embodiment, the base of each well is formed from a circular platelet of 8 mm diameter carrying the array 60 of test structures. Each such platelet is cut out of a 3 mm thick 6 inch injection moulded wafer manufactured according to the process described below. Other formats of sample container can also be used, for example Millipore EZ-ChIP (trademark) cell format.

As mentioned, the base of each well of the microtiter plate can be provided with an array 60. As an alternative, not all of the wells may be provided with such an array. The arrays can be the same as between each such well, or can be different between at least some of the wells. In an embodiment, the arrays can be the same as between all of the wells. Accordingly, the shading employed in FIG. 2 is to be taken as purely schematic, and may be representative of a set of identical arrays or a set of arrays in which at least some of the arrays are different.

A particular substrate with multiple differently structured test areas can be manufactured, if desired in large quantities, using an injection moulding process as now described.

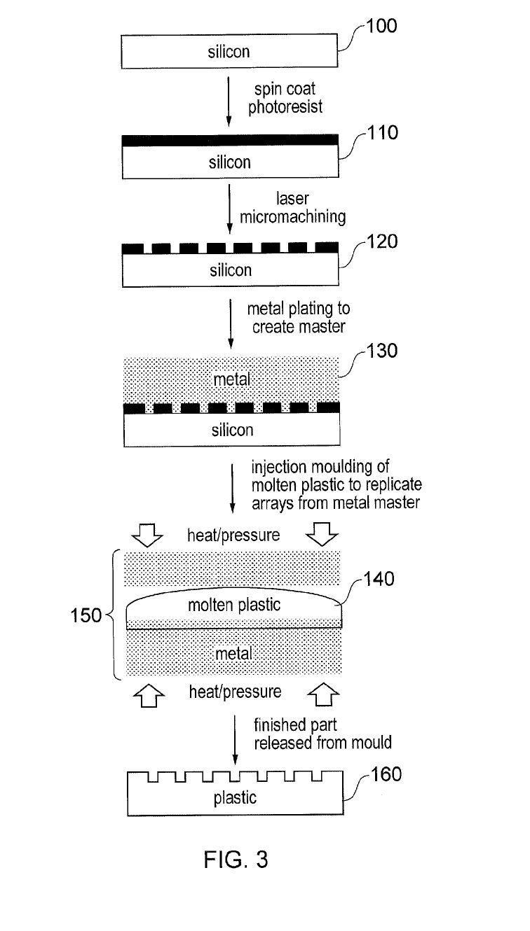

FIG. 3 schematically shows the principal steps in a substrate manufacturing process.

The first part of the process is to manufacture a master or die. This is because the basis of the fabrication technique is to mould a substrate using a master die so that a surface of the substrate includes one or more formations complementary to respective moulding formations on the die.

A silicon or glass wafer 100 is spin coated with a photoresist so as to create a photoresist coated substrate 110. An excimer laser or other suitable light source (not shown) is then used to expose the photoresist to define a structure with high spatial resolution, for example by direct laser micromachining. In this process, the material to be exposed is transparent to the laser light used. However, in the focal volume of this highly focused laser beam chemical or physical modification is created. Ultimately a selective solubility of the exposed area relative to the surrounding is achieved. In a developer bath, depending on the used photosensitive material exposed or unexposed areas are removed. Thus, almost any “2.5D” structures from a variety of photosensitive materials can be realized (for example SU-8 or the positive photoresist AZ9260 from AZ Electronic Materials are examples of suitable types of photoresist). Note that the expression “2.5D” is notation to indicate a three-dimensional structure which is limited by the fact that undercut formations cannot be implemented by this technique.

Alternative technologies for structuring the resist master are e-beam lithography or mask based lithography processes. Laser write Lithography can also be used with inorganic phase transition materials instead of the photoresist pushing the size resolution limit below the wavelength of the laser. Further details of applicable processes can be found in JP4274251 B2 (=US2008231940A1) and JP 2625885 B2 (no English equivalent). Further background documents relating to the fabrication process for microfluidic devices include: Bissacco et al, “Precision manufacturing methods of inserts for injection moulding of microfluidic systems”, ASPE Spring Topical Meeting on Precision Macro/Nano Scale Polymer Based Component & Device Fabrication. ASME, 2005; Attia et al, “Micro-injection moulding of polymer microfluidic devices”, Microfluidics and Nanofluidics, vol. 7, no. 1, July 2009, pages 1-28; and Tsao et al, “Bonding of thermoplastic polymer microfluidics”, Microfluidics and Nanofluidics, 2009, 6:1-16. All of these documents are hereby incorporated by reference.

An example of the resulting coated substrate is shown as a substrate 120.

Once the photoresist has been suitably structured and the exposed (or non-exposed) material removed, a metal plating processing step is applied. Electroplating is used to deposit a nickel layer by electrolysis of nickel salt-containing aqueous solutions, so-called nickel electrolytes. Nickel electrolytes usually have nickel or nickel pellets as the anode. They serve the supply of metal ions. The process for the deposition of nickel has long been known and been highly optimized. Most nickel electrolytes to achieve an efficiency of >98%, which means that over 98% of the current supplied to be used for metal deposition. The remaining power is lost in unwanted electrolytic processes, such as hydrogen. The transcription of lithographically structured micro-features is strongly dependent on compliance with the correct parameters. The continuous supply of additives, but also the metal ion content and the temperature and the pH value needs to be.

The result is a metal version 130 of the structure defined by the partially removed photoresist.

This electroplating process can be repeated either to make multiple copies of the same master from the silicon or to create a negative copy from the first metal stamper that is produced from the silicon.

Direct milling into steel can be used as an alternative to silicon and photoresist in order to master such microstructures. Other methods, or other variations on the methods described above, are also possible, as described in the documents referenced below.

Many interesting microstructures are in the size of 500 nm to several micrometres, so that cells can interact with the microstructures' protrusions directly.

The master is then used as part of a mould in an injection moulding process to create the structured surfaces in polymer. In an injection moulding machine, polymers are plasticized in an injection unit and injected as molten material 140 into a mould 150. The cavity of the mould (including the master as discussed above) determines the shape and surface texture of the finished part 160. The polymer materials need to be treated carefully to prevent oxidation or decomposition as a result of heat or sheer stresses. Heat and pressure are applied to press molten polymer onto the structured surface of the master. After a suitable filling, cooling and hardening time, the finished structure 160 is ejected from the mould. The surface quality of the component can be selected almost arbitrarily enabling a wide variety of micro- and nano-structured test areas to be formed in an array.

The cost of the master and the larger moulding tool it will form a part of represents a large part of the total necessary investment, so the process lends itself to high volumes. Simple tools enable economic viable prototyping from a threshold of a few thousand parts. Tools for production can be used up to make up to several million parts.

Suitable polymers for the container include: polystyrene (PS), polypropylene (PP), polyethylene (PE), cycloolefin (co-) polymer (COP), styrene-acrylonitrile copolymer (SAN), polyamide (nylon), polyimide (PI), polycarbonate (PC), and polymethyl methacrylate (PMMA). Example plastics compounds we have tested in detail and have shown good results are as follows. PS: BASF ‘158K’ which is a high heat, clear material suitable for injection moulding. COP: Zeon Chemicals ‘Zeonor 1060R’ which is a clear, low water absorption material suitable for injection moulding. PMMA: Asahi Kasei ‘Delpet 70NH’ which is transparent and suitable for injection moulding. PP: Lyondell Basell Industries ‘Purell HM671T’.

The injection moulded substrate can be further processed to add further modifications to the test areas by varying the surface properties in a test area specific manner.

One surface property that can be controlled and modified is surface potential which can be given different values in different ones of the test areas. For example a plurality of test areas with the same micro- or nano-structuring may be treated to have systematically varying surface potential values. The surface potential value can be varied by applying different amounts or types of plasma treatment. Plasma techniques are especially useful because they can deposit ultra thin (a few nm), adherent, conformal coatings. Glow discharge plasma is created by filling a vacuum with a low-pressure gas (for example argon, ammonia, or oxygen). The gas is then excited using microwaves or current which ionizes it. The ionized gas is then thrown onto a surface at a high velocity where the energy produced physically and chemically changes the surface. After the changes occur, the ionized plasma gas is able to react with the surface to lower the surface energy. In oxygen plasma the surface becomes more hydrophilic as the carbons in the plastic are oxidized. Plasma polymerization is a special variant of the plasma-activated chemical vapor deposition (PE-CVD) specifically suitable for providing biocompatible surfaces. During plasma polymerization vaporized organic precursors (precursor monomers) are activated in the process chamber by a plasma initially. Activation caused by the ionized molecules which are formed already in the gas phase result first in molecular fragments. The subsequent condensation of these fragments on the substrate surface then causes under the influence of substrate temperature, electron and ion bombardment, the polymerization and thus the formation of a closed plasma polymerized layer. The structure of the emerging “plasma polymer” is comparable to highly cross-linked thermosets, because they form a largely random covalent network. Such a layer can be hydrophilic and water stable at the same time, and thus show good adhesion for cells.

Corona treatment (sometimes referred to as air plasma) is a surface modification technique that uses a low temperature corona discharge plasma to impart changes in the properties of a surface. A linear array of electrodes is often used to create a curtain of corona plasma. Corona treatment is a widely used surface treatment method in the plastic films and parts. It represents a cost effective way of providing a surface suitable for cell adhesion.

The amount of plasma treatment can be dosed by the energy or power applied, for example 200 W, 300 W, 400 W, 500 W, 800 W and so on.

A further surface property that can be controlled and modified is by including a coating which is selectively applied to only some of the test areas and/or is applied differently from test area to test area.

For example, sputtering may be used to deposit a coating. Sputter deposition is a physical vapor deposition (PVD) method of depositing thin films by sputtering, that is ejecting, material from a “target,” that is source, which then deposits onto the substrate. At higher gas pressures, the ions collide with the gas atoms that act as a moderator and move diffusively, reaching the substrates or vacuum chamber wall and condensing after undergoing a random walk. The sputtering gas is often an inert gas such as argon. The availability of many parameters that control sputter deposition make it a complex process, but also allow experts a large degree of control over the growth and microstructure of the film. This is why sputter coating is often used to provide cell-growth compatible coatings.

Other coating examples include a coating of: a protein layer, a ligand, an amine and/or a liquid crystal.

The combined effect of the surface potential, coating and the micro- or nano-structuring can be defined and measured in terms of contact angle, or in other words the degree of hydrophobicity or hydrophilicity of the surface.

According to the manner in which the structures are being used, the finished part 160 (optionally as processed according to one or more of the processing techniques discussed above) may form one array of the type shown in FIG. 1, or more than one such array, or a set of individual arrays 60 of the type shown in FIG. 2, or another arrangement. If the finished part 160 includes more than one array, or includes extra material, for example around the edge of an array, then the finished part can be cut or cleaved to part(s) of the required size(s) using known techniques for cutting polymer parts.

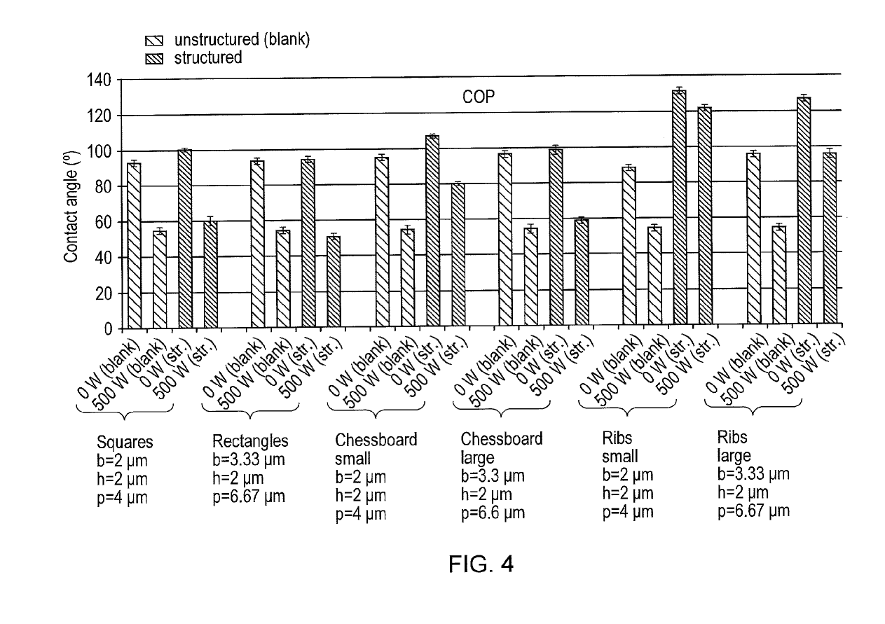

FIG. 4 shows some comparative results of contact angle measured on various structured surfaces formed on COP. The COP in this example is Zeonor 1060R. The comparisons are between larger and smaller versions of the same or similar structure patterns, between surfaces which have and have not been plasma treated, and between each structured surface and a flat unstructured area of the same plastics compound. Note, of course, that because the same COP material is used for all the tests, the measurements for the unstructured (blank) samples are identical across each set of results.

The structure patterns are examples of those shown schematically in FIG. 1. These include square shaped patterns, rectangular shaped patterns, chessboard patterns (alternating squares in a manner similar to a chessboard) and ribbed patterns.

The plasma treatment is a 500 W corona treatment of 1 hour duration. The measurements corresponding to no plasma treatment are indicated as “0 W”.

The measurements are shown as vertical bars corresponding to a vertical scale representing contact angle. Error bars 200 schematically indicate the measurement errors associated with each respective measurement.

Some general effects of plasma treatment compared to no plasma treatment, and structure compared to no structure are apparent by comparing the contact angle within each group of 4 measurements on a given test sample. However, the comparison which is highlighted here and which is most relevant for the present technology is the effect of structure size which can be seen by comparing the structured results for different sizes of the same structure pattern: namely comparing the “ribs large” group with the “ribs small” group, the “chessboard large” group with the “chessboard small” group, and the “squares” group with the “rectangles” group (rectangles being elongate expansions of the squares). For example, the rightmost result which is ribs-large-500 W-structured, this has a contact angle of about 95°. This is to be compared with the equivalent result for ribs-small which shows a contact angle of slightly above 120°. In all such comparisons between different sizes of the same pattern there are significant differences in contact angle.

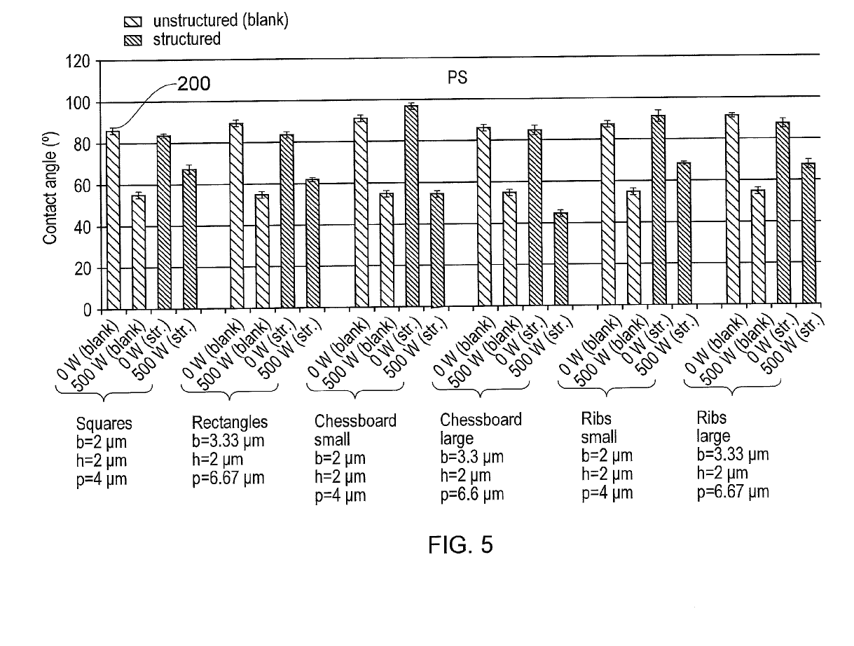

FIG. 5 shows some comparative results of contact angle measured on various structured surfaces formed on PS. The respective patterns and plasma treatments are directly comparable on a one-to-one basis with the COP results. The PS used for the measurements of FIG. 5 is BASF 158K. The discussion points are the same as for the COP results. Once the culturing container has been provided it can be used for testing the culturing of any particular animal cells under a particular set of growth conditions, where of course the growth is tested simultaneously on each of the test areas. According to embodiments of the technology, this provides an example of providing a test sample container with a substrate having a surface subdivided into a plurality of test areas, each with a pre-defined combination of surface properties including a micro- or nano-structure, wherein said micro- or nano-structure has at least one dimensional parameter whose value is different in different ones of the test areas so as to have different test areas that cover a range of values of the or each said dimensional parameter.

The results are then analyzed on a test area by test area basis using an appropriate technique which itself is preferably parallel, or in other words a technique which allows simultaneous analysis of all of the test areas. Various optical analysis methods will be suitable such as known microscopy or spectroscopy techniques, for example confocal microscopy or mass spectrometry. A simple measurement is one of optical density, for example transmissivity or absorption, which is a measure of how much cell growth has occurred over the test area. Appropriate staining or other tagging may be used. An alternative to optical analysis is the use of mass spectroscopy analysis to detect properties of the results at each test area. The analysis results can be compared by a data processing apparatus arranged to process electrical or other signals indicative of the results associated with each test area and to select a number (for example, a predetermined number, or that number for which the results reach and/or exceed a predetermined parameter) of test areas.

Example tests have been performed in respect of embodiments of the present techniques by growing the NTera2 cell line onto the example structures. NTera2 is a pluripotent human embryonal carcinoma (EC) stem cell line which shares many characteristics with human embryonic stem cells (hESCs). The tests included absorption or luminescence measurements of the cells and colonies to measure how well the cells had cultured. Cell culturing initiates with adhesion of a single (stem) cell onto a surface location. There then follows a period of proliferation during which a colony grows from the initially anchored cell. In the case of stem cells, at a certain point differentiation may occur. If differentiation is the goal of the culturing, then this should occur ideally as soon as possible and as evenly as possible. Another interesting effect which can sometimes be observed is motility, which is the movement of a cell along the surface after adhesion.

Since all of these behaviours are dependent on each other they can also be considered as a natural sequence: in other words, the better the cell adhesion, the less the cells need to duplicate before they form a confluent layer and the earlier they can be processed/analysed. Moreover, the more naturally the cells adhere to a surface the earlier they will start to move around, without being lost in the medium.

The existing literature on substrates considers speed of proliferation as the main specification to optimize, since it is always desired to be able to grow colonies as quickly as possible to reduce overall experiment time.

However, the present experiments show the principal effect of structured surfaces, or more precisely, the biggest differences between different types and dimensions of structured surfaces which are observed relate to changes in the onset of differentiation and changes in the motility. In the tests, proliferation times seem largely independent of structure. Moreover, in the present tests adhesion is seen to be structure dependent, but structuring seems to mildly hinder adhesion rather than promote it compared to smooth, unstructured surfaces. Moreover, some correlation of contact angle and adhesion is observed. Further, it is found that motility is strongly promoted in certain rib structures in a rib-dimension dependent manner with migration speeds of 10-40 micrometres per second being observed.

These steps therefore provide an example of test culturing particular cells or test binding particular proteins under particular experimental conditions simultaneously on each of the test areas and analyzing the cultured cells or the bound proteins on a per test area basis.

Based on the analysis, the user (or an automated selection arrangement such as a data processing apparatus) can select one of the test areas as providing suitable, for example the best (or at least better than some others), culturing conditions for the particular animal cells under the particular growth conditions targeted to the parameter or parameters which are most important to optimize or at least improve for the particular study, for example adhesion, proliferation, differentiation or motility. This can be done by systematic variation of structure sizes of the same patterns, or simply by testing very large numbers of structured surfaces in parallel with different patterns and structure sizes. Moreover, if a number of test wafers or well plates are available, each with a large number of test structures, plasma treatment may be varied from wafer to wafer or well plate to well plate, for example a dosage or exposure increasing from 100 W to 1000 W may be carried out in steps of 100 W. Overall, this represents an example of selecting based on said analyzing one of the test areas as being suitable for culturing the particular cells or binding the particular proteins under the particular experimental conditions.

As part of a method of manufacturing a batch of sample containers optimized for culturing particular cells or binding particular proteins under particular experimental conditions, the user can then request manufacture of or supply of a previously manufactured batch of culturing containers with one or more production test areas, each of which has the surface properties of the test area shown by the test culturing to have the best properties for the program to be undertaken. Such a batch represents an embodiment of the present technology.

Manufacture of the batch can take place using, in essence, the same technique as described with reference to FIG. 3. However, it is noted the format of the batch container may be different from the test container as desired. For example, the test container may be essentially flat, for example being the ‘virtual’ microtiter plate mentioned above, whereas the batch container may be in a conventional microtiter plate format with any desired numbers of wells, such as 6, 24, 48, 96, 384, 1536 and so on, wherein the micro- or nano-structured test area covers an interior portion of the base of each well. This therefore represents an example of manufacturing and/or supply of a batch of sample containers with one or more areas, each of which has the surface properties of said selected test area from the test culturing or test binding.

A test area can thereby be selected to identify surface structures which either stimulate adherence, proliferation or differentiation of cell, to enable selection the surface which is most beneficial for a specific cell line in a specific experiment. For example, it has been shown that neuronal stem cell adhere faster on linear ridges (triangle profile with 1 μm in depth and 1 μm in pitch), stretch along the ridges and proliferate in the longitudinal direction of the ridges. These cells move with reproducible speed along the surface structures. Thus the experiments with different cells can be accelerated. An in vivo parameter (cell functionality/viability) can be measured. An assay can be developed with measure the influence on the mobility of such cells. The resulting protocols could be offered to wider user base. The cell culture lines thereby differentiating from stem cell lines could be offered.

In some embodiments, deep structures can be provided on the surface to allow reagent diffusion to the bottom of the cell body. Such selection plates can be combined with various surface coatings. The surface may be chemically modified or plasma oxidized. Furthermore bio-plastics or sponge like polymers might be used which proved appropriate cofactors for cell function. Certain areas of the selection plates may be structured or coated so that there is no cell adhesion and the cells can be analyzed automatically.

The above discussion has concentrated on culturing animal cells, in particular stem cells, but the present technology can also be applied to selection of suitable substrates to promote protein binding or functionality. Proteins, especially in their native configuration, also show differing and often unexpected types of behaviour if adsorbed onto surfaces. The more natural the environment (that is to say, the more similar to the in vivo environment), the more an in vitro test or experiment will give information about the in vivo behaviour. Proteins will show varying adhesion properties depending on the structure surface and treatment, being more or less likely to retain their native configuration and stay adsorbed to a surface depending on the properties of the surface, including its structure and treatment. For example it is known that different protein adsorption mechanisms lead to different levels of functionality and native conformation of the proteins bound to the surfaces. Typically, the surface is provided with a protein binding membrane which serves to bind proteins from solution while retaining their native condition. Available membranes have very different chemical composition and surface structure. The most widely used materials are porous nitrocellulose, nylon and polyvinylidene fluoride (PVDF) membranes. While having different chemical compositions, these membranes have in common that they provide hydrophobic pockets in a generally hydrophilic surfaces. In addition to the various surface chemistries, surface microstructures can serve to increase the overall surface area and bind more protein per unit area. Such micro-structured surfaces can be manufactured with established injection moulding technologies as described above and combined with chemical surface modifications. Metal coating (for example Au) can be included. For the researcher it is most difficult to choose the more suitable among the many commercial suppliers of protein binding surfaces.

In some embodiments, a single protein binding membrane extends over a substrate having an array of test areas with different micro- or nano-structuring. In other embodiments, different protein binding membranes are arranged in different test areas or groups of test areas. For example, different commercially available protein binding membranes can be applied to the different individual wells or groups of wells of one multi-well plate. In this way, each different commercially available membrane can be tested with one or more different micro- or nano-structured surfaces in one or more respective wells. This allows a user to test protein adsorption in a simple and reproducible way on several, preferably all major, commercially available protein binding membranes. The relevant membranes, ideally in large quantities, are stocked ready for direct supply to the customer.

Further embodiments therefore include a test binding container with a substrate having a surface subdivided into a plurality of test binding areas and optionally coated with a protein binding membrane which is used to bind particular proteins under particular conditions simultaneously on each of the test areas. Analysis is then performed on a per binding area basis and based on that analysis, for example a measurement of how many proteins have bound to each area or a measurement of the functionality of the proteins which have bound to each area, one of the binding areas is selected as providing suitable conditions for binding the particular protein under the particular binding conditions. A batch of protein binding containers is then manufactured with one or more production protein binding areas, each of which has the surface properties of said selected area from the test.

The test selection containers are thus used to identify a material and structure combination which optimizes a suitable property adhesion or functionality of the protein. Each test selection container can be used to test simultaneously a variety of structure and material combinations to select one which is most beneficial for a specific protein experiment.

The manufacturing processes used to manufacture the test container are preferably ready for volume manufacturing without modification so that substrates can be manufactured in larger quantities safe in the knowledge that its surface properties will be the same as in the selected test area of the test container.

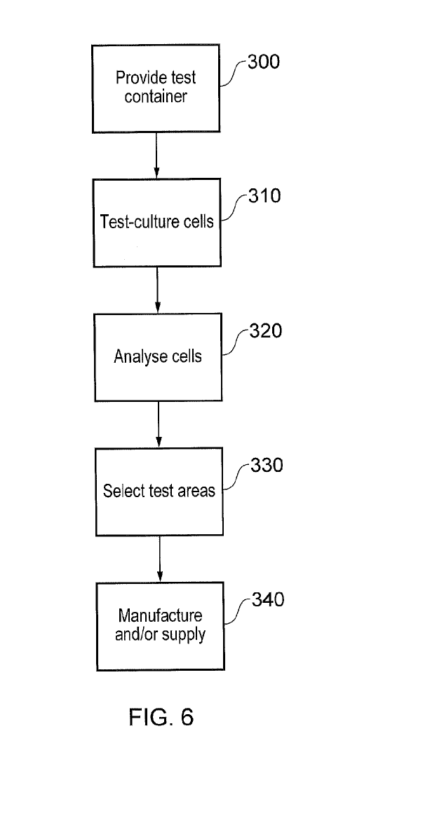

FIG. 6 is a schematic flowchart showing an example process according to the present technology for manufacturing a batch of sample containers optimized (or at least targeted at least in part) for culturing particular cells or binding particular proteins under particular experimental conditions. According to FIG. 6 an example of such a method comprises the following steps.

At a step 300, providing a test sample container with a substrate having a surface subdivided into a plurality of test areas, each with a pre-defined combination of surface properties including a micro- or nano-structure, wherein said micro- or nano-structure has at least one dimensional parameter whose value is different in different ones of the test areas so as to have different test areas that cover a range of values of the or each said dimensional parameter.

At a step 310, test culturing particular cells or test binding particular proteins under particular experimental conditions simultaneously on each of the test areas.

At a step 320, analyzing the cultured cells or the bound proteins on a per test area basis;

At a step 330, selecting based on said analyzing one of the test areas as being suitable for culturing the particular cells or binding the particular proteins under the particular experimental conditions.

At a step 340, manufacturing and/or supply of a batch of sample containers with one or more areas, each of which has the surface properties of said selected test area from the test culturing or test binding.

Further respective aspects and features of embodiments of the present technology are defined by the following numbered clauses:

1. A method of manufacturing a batch of sample containers optimized for culturing particular cells or binding particular proteins under particular experimental conditions, the method comprising:

providing a test sample container with a substrate having a surface subdivided into a plurality of test areas, each with a pre-defined combination of surface properties including a micro- or nano-structure, wherein said micro- or nano-structure has at least one dimensional parameter whose value is different in different ones of the test areas so as to have different test areas that cover a range of values of the or each said dimensional parameter;

test culturing particular cells or test binding particular proteins under particular experimental conditions simultaneously on each of the test areas;

analyzing the cultured cells or the bound proteins on a per test area basis;

selecting based on said analyzing one of the test areas as being suitable for culturing the particular cells or binding the particular proteins under the particular experimental conditions; and

manufacturing and/or supply of a batch of sample containers with one or more areas, each of which has the surface properties of said selected test area from the test culturing or test binding.

2. The method of clause 1, wherein each test area is isolated from each other test area by a cytophobic area.
3. The method of clause 2, wherein the cytophobic area is an unstructured surface portion which is hydrophobic.
4. The method of clause 2 or 3, wherein the unstructured surface portion is substantially co-planar with the test areas.
5. The method of clause 1, wherein each test area is isolated from each other test area by the test areas being formed as isolated wells recessed beneath an upper surface level with interconnecting sidewalls.
6. The method of any preceding clause, wherein the dimensional parameter includes a pitch of a periodic feature of the micro- or nano-structure.
7. The method of any preceding clause, wherein the dimensional parameter includes a depth of the micro- or nano-structure.
8. The method of any preceding clause, wherein the surface properties include surface potential which has a different value in different ones of the test areas.
9. The method of clause 8, wherein the surface potential value is varied by applying different amounts or types of plasma treatment.
10. The method of any preceding clause, wherein the surface properties include a coating which is selectively applied to only some of the test areas and/or is applied differently from test area to test area.
11. The method of clause 10, wherein one or more of the growth surfaces include a coating of: a protein layer, a ligand, an amine and/or a liquid crystal.
12. The method of any preceding clause, wherein said analyzing involves an optical analysis method.
13. The method of any preceding clause, wherein said analyzing involves mass spectroscopy.
14. A batch of sample containers manufactured according to the method of any one of the preceding claims.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be apparent that numerous modifications and variations of the present disclosure are possible in light of the above teachings. It is therefore to be understood that within the scope of the appended claims, the technology may be practiced otherwise than as specifically described herein.

### REFERENCES

1. Théry Journal of Cell Science 123, 4201-4213 (2010).
2. Hu et al J Vac Sci Technol B vol. 23(6), pages 2984-2989 (2005)
3. US20080241926A1 Lee et al
4. U.S. Pat. No. 6,306,646 Saad et al
5. Lee et al IPCBEE vol. 2 (2011) IACSIT Press
6. US20090220561A1
7. U.S. Pat. No. 5,173,421 Kiniwa et al
8. Vogt et al Biotechnol. Prog. 2003, 19, 1562-1568
9. EP1199354A1
10. US20020095219A1 Nelles et al
11. Intelligent Substrates website: http://www.intelligentsubstrates.com/
12. US20050208467A1 Qiu
13. US20100136647A1 Algotsson
14. U.S. Pat. No. 3,910,819 Rembaum
15. U.S. Pat. No. 7,041,343 Nelles

1. A method of manufacturing a batch of sample containers optimized for culturing particular cells or binding particular proteins under particular experimental conditions, the method comprising:
providing a test sample container with a substrate having a surface subdivided into a plurality of test areas, each with a pre-defined combination of surface properties including a micro- or nano-structure, wherein said micro- or nano-structure has at least one dimensional parameter whose value is different in different ones of the test areas so as to have different test areas that cover a range of values of the or each said dimensional parameter;
test culturing particular cells or test binding particular proteins under particular experimental conditions simultaneously on each of the test areas;
analyzing the cultured cells or the bound proteins on a per test area basis;
selecting based on said analyzing one of the test areas as being suitable for culturing the particular cells or binding the particular proteins under the particular experimental conditions; and
manufacturing and/or supply of a batch of sample containers with one or more areas, each of which has the surface properties of said selected test area from the test culturing or test binding.

2. The method of claim 1, wherein each test area is isolated from each other test area by a cytophobic area.

3. The method of claim 2, wherein the cytophobic area is an unstructured surface portion which is hydrophobic.

4. The method of claim 2, wherein the unstructured surface portion is substantially co-planar with the test areas.

5. The method of claim 1, wherein each test area is isolated from each other test area by the test areas being formed as isolated wells recessed beneath an upper surface level with interconnecting sidewalls.

6. The method of claim 1, wherein the dimensional parameter includes a pitch of a periodic feature of the micro- or nano-structure.

7. The method of claim 1, wherein the dimensional parameter includes a depth of the micro- or nano-structure.

8. The method of claim 1, wherein the surface properties include surface potential which has a different value in different ones of the test areas.

9. The method of claim 8, wherein the surface potential value is varied by applying different amounts or types of plasma treatment.

10. The method of claim 1, wherein the surface properties include a coating which is selectively applied to only some of the test areas and/or is applied differently from test area to test area.

11. The method of claim 10, wherein one or more of the growth surfaces include a coating of: a protein layer, a ligand, an amine and/or a liquid crystal.

12. The method of claim 1, wherein said analyzing involves an optical analysis method.

13. The method of claim 1, wherein said analyzing involves mass spectroscopy.

14. A batch of sample containers manufactured according to the method of claim 1.
